(19) United States
(12) Patent Application Publication
(10) Pub. No.: US 2026/0294849 A1
(43) Pub. Date: Oct. 1, 2026

(54) METHODS FOR TREATING CORNEAL INJURIES

(71) Applicant: National Taiwan University, Taipei City (TW)

(72) Inventors: Sung-Jan LIN, Taipei City (TW); Yueh-Feng WU, Taipei City (TW)

(21) Appl. No.: 19/164,213

(22) PCT Filed: Apr. 3, 2024
(86) PCT No.: PCT/CN2024/085758

§ 371 Date: Sep. 11, 2025

Related U.S. Application Data
(60) Provisional application No. 63/458,095, filed on Apr. 8, 2023.

(51) Int. Cl. A61K 31/167; A61K 9/00; A61K 31/185; A61K 31/7032; A61K 31/7036; A61P 27/02

(52) U.S. Cl. CPC A61K 31/167; A61K 9/0048; A61K 31/185; A61K 31/7032; A61K 31/7036; A61P 27/02

(57) ABSTRACT

Provided herein is a method for treating corneal injury in a subject. Specifically, the method includes administering to the subject an effective amount of a compound of Formula (I) or its pharmaceutically acceptable salt, so as to ameliorate symptoms associated with corneal injury,

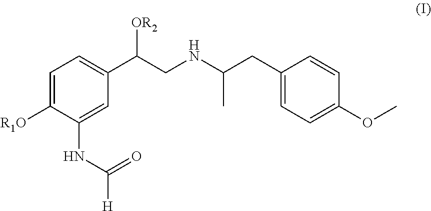

wherein
R₁ is H, —SO₃H, or

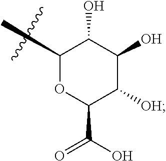

and
R₂ is H or

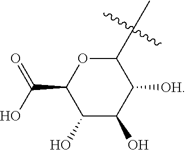 [SMILES: *C1O[C@H](C(=O)O)[C@@H](O)[C@H](O)[C@H]1O]

### CROSS REFERENCE TO RELATED APPLICATIONS

This application is a national stage filing under 35 U.S.C. 371 from International Patent Application Serial No: PCT/CN2024/085758 filed Apr. 3, 2024, and published on Oct. 17, 2024, which claims the benefit of U.S. Provisional Application No. 63/458,095, filed Apr. 8, 2023; the entireties of which are incorporated herein by reference.

### BACKGROUND OF THE INVENTION

### 1. Field of the Invention

The present disclosure in general relates to a novel treatment for corneal injury.

### 2. Description of Related Art

The cornea is one of the transparent layered tissues that constitute the eyeball. In terms of human, the cornea has a diameter of about 12 mm, a thickness of the central part of about 0.5 mm, and a thickness of the peripheral part of about 0.7 mm, and is composed of five layers of corneal epithelium, Bowman's membrane, stroma of the cornea, Descemet's membrane, and corneal endothelium from the surface. Among the five layers that compose of the cornea, corneal endothelial cells are responsible for regulating the water content and thickness of the cornea constant, thereby maintaining the transparency of the cornea, and lack of corneal endothelial cells leads to corneal opacity and causes a significant decrease in the visual acuity.

The cornea may be injured physically (e.g., external force, eye surgery, laser, etc.), chemically (e.g., exposure to harmful chemicals), or biologically (e.g., infection by microbes or suffering from some corneal diseases). As the corneal injury deteriorates to a certain extent, a conventional corneal transplantation therapy is inevitably needed. Nonetheless, there are approximately 185,000 corneal transplants are performed annually in 116 countries, and a total of 284,000 corneas are procured from 742 eye banks, which is far from enough to supply an estimated 12.7 million patients for corneal transplantation globally. As such, factors such as a shortage of donors who can provide the cornea needed for transplantation, a rejection reaction to the donor-derived cornea that may occur, and the like, limit the scope of application of the corneal transplantation therapy.

On the other hand, common indications that require the corneal transplantation therapy include keratoconus and corneal endothelial decompensation, which account for more than 85% of the cases; the keratoconus and corneal endothelial decompensation usually occurs after intraocular surgery, in which the intraocular surgery causes the corneal injury (i.e., sudden loss of corneal endothelial cell density) and in turn exacerbates the condition of the keratoconus and corneal endothelial decompensation. However, it is unclear about the response of the corneal endothelial cells to injury, and making it a great challenge to prevent postoperative corneal decompensation.

In view of the foregoing, there exists in the related art a need for a novel treatment method for treating corneal injury, to improve the wound healing of the cornea (such as after intraocular surgery), which is via preventing the corneal decompensation, so as to reduce the need for corneal transplantation surgery.

### SUMMARY

One aspect of the present disclosure aims at providing a novel treatment for corneal injury. Thus, as embodied, and broadly described herein, the present disclosure relates to a method for treating corneal injury in a subject. The method comprises administering to the subject an effective amount of a compound or its pharmaceutically acceptable salt, wherein the compound is selected from the group consisting of a compound of Formula (I), Bhilawanol A, Encainide, Fenbutrazate, Levonordefrin, Permethrin, and 2,2,4,4-Tetrahydroxybenzophenone (THBP),

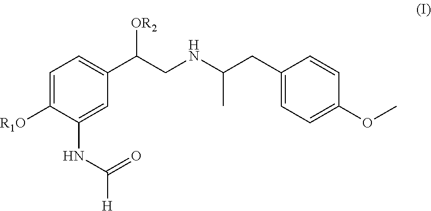

wherein
R₁ is H, —SO₃H, or

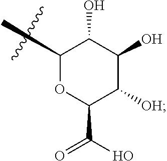

and
R₂ is H or

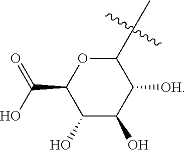 [SMILES: *C1O[C@H](C(=O)O)[C@@H](O)[C@H](O)[C@H]1O]

According to embodiments of the present disclosure, in the compound of Formula (I),
R₁ and R₂ are independently H;
R₁ is —SO₃H, and R₂ is H;
R₁ is H, and R₂ is

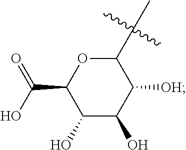 [SMILES: *C1O[C@H](C(=O)O)[C@@H](O)[C@H](O)[C@H]1O]

or
R₁ is

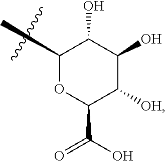

### and R₂ is H.

According to embodiments of the present disclosure, the compound of formula (I) is in the form of a salt. In some embodiments, the compound of formula (I) is in the form of a fumarate salt. In other embodiments, the compound of formula (I) is in the form of a tartrate salt.

According to preferred embodiments of the present disclosure, said corneal injury is a corneal endothelial injury. In some embodiments, the corneal injury is caused by intraocular surgery. In other embodiments, the corneal injury is resulted from apoptosis during wound healing.

According to the embodiments of the present disclosure, the compound of the present disclosure may be administered in the amount ranging from about 0.1 ng/kg to about 1 ag/kg.

As could be appreciated, an ophthalmological anti-infective agent may be used in the present method in combination with the present compound to improve wound healing of the cornea. The ophthalmological anti-infective agent may be administered to the subject prior to, in conjunction with, or subsequent to application of the present compound to the subject. Non-limiting examples of the ophthalmological anti-infective agent include, but are not limited to, Chloramphenicol, Azidamfenicol, Tetracycline, Chlortetracycline, Oxytetracycline, Dihydrostreptomycin, Neomycin, Framycetin, Kanamycin, Amikacin, Tobramycin, Gentamicin, Netilmicin, Micronomicin, Erythromycin, Natamycin, Ampicillin, Benzylpenicillin, Polymyxin B, Tyrothricin, Rifamycin, Fusidic acid, Sulfamethizole, Sulfafurazole, Sulfadicramide, Sulfacetamide, Sulfafenazol, Idoxuridine, Trifluridine, Aciclovir, Interferon, Vidarabine, Famciclovir, Fomivirsen, Ganciclovir, Nitrofural, Bibrocathol, Resorcinol, Sodium borate, Hexamidine, Chlorhexidine, Sodium propionate, Ofloxacin, Norfloxacin, Ciprofloxacin, Dibrompropamidine, Propamidine, Picloxydine, Lomefloxacin, Povidone-iodine, Levofloxacin, and Gatifloxacin. Also, to achieve better therapeutic effect, the present compound is administered intracamerally to the subject.

In all embodiments of the present disclosure, the subject suitable for receiving treatment of the present method is a mammal, preferably, a human.

Many of the attendant features and advantages of the present disclosure will becomes better understood with reference to the following detailed description considered in connection with the accompanying drawings.

### BRIEF DESCRIPTION OF THE DRAWINGS

These and other features, aspects and advantages of the present invention will become better understood with reference to the following description, appended claims and the accompanying drawings, where:

FIG. 1 depicts the response of the corneal endothelial cells after the laser ablation treatment in accordance with one embodiment of the present disclosure;

FIG. 2 depicts the wound healing status of the laser-injured corneal endothelial cells after treating the present compound in accordance with one embodiment of the present disclosure; and

FIG. 3 depicts the density of the laser-injured corneal endothelial cells on Day 2 after treatment with the present compound in accordance with another embodiment of the present disclosure, in which *, P<0.05.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of cell biology and pharmacology, which are within the skill of the art. Such techniques are explained fully in the literature.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “cornea” refers to a transparent membrane on the surface of the eyeball in the middle region of the eye, which has a structure enabling the protection of the eye from the outside and allowing the light to pass through and refract, thereby allowing visual acuity.

The term “corneal injury” as used herein refers to a condition where the cornea is damaged at any parts thereof that may be treatable with the methods as described herein, in which the parts of the cornea include, but are not limited to, the layer of the corneal epithelium, the Bowman's membrane, the stroma of the cornea, the Descemet's membrane, or the corneal endothelium; and the damage may be caused physically (e.g., trauma, intraocular surgery, contact lenses usage, ultraviolet (UV) radiation, etc.), chemically (e.g., chemical exposure), or biologically (e.g., infections).

As used herein, the term “corneal disease” refers to all of the diseases or damage that cause damage to the cornea, thereby causing a loss of transparent vision. Specifically, the corneal disease may be any one selected from the group consisting of dry eye syndrome, hyperemia, corneal angiogenesis, and keratitis, but the corneal disease is not limited thereto and any disease that may accompany hyperemia or may occur due to the corneal angiogenesis may be included without limitation.

The terms “treatment” and “treating” as used herein may refer to a curative or palliative measure. In particular, the term “treating” as used herein refers to the application of the present method (or administration of any compound as set forth in the present method) to a subject, who has corneal injury or a symptom associated with corneal injury, with the purpose to partially or completely alleviate, ameliorate, relieve, delay onset of, inhibit progression of, reduce severity of, and/or reduce incidence of one or more symptoms or features of corneal injury.

The terms “administered,” “administering,” or “administration” are used interchangeably herein to refer means either directly administering at least one compound as set forth in the present method, or administering a pharmaceutical composition comprising the same, to the subject.

The term “an effective amount” as used herein refers to an amount effective, at dosages, and for periods of time necessary, to achieve the desired therapeutically desired result with respect to the treatment of corneal injury or symptoms associated with corneal injury in a subject. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), the specific formulations employed, and the specific route of administration and like factors within the knowledge and expertise of the health practitioner. Effective amount may be expressed, for example, in grams, milligrams or micrograms or as milligrams per kilogram of body weight (mg/kg). Alternatively, the effective amount can be expressed in the concentration of the active component (e.g., the compound as set forth in the present method), such as molar concentration, mass concentration, volume concentration, molality, mole fraction, mass fraction and mixing ratio. Persons having ordinary skills could calculate the human equivalent dose (HED) for the medicament (the compound as set forth in the present method) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “a pharmaceutically acceptable salt” refers herein as a salt which is formed by the interaction of a base (e.g., the compound of formula (I) in the present disclosure) with a pharmaceutically acceptable acid, including organic or inorganic types of acids such as hydrochloric acid, hydrobromic acid, hydroiodic acid, perchloric acid, methylsulfonic acid, sulfuric acid, nitric acid, phosphoric acid, acetic acid, propionic acid, glycolic acid, pyruvic acid, malonic acid, succinic acid, maleic acid, fumaric acid, tartaric acid, citric acid, benzoic acid, carbonic acid, cinnamic acid, methanesulfonic acid, ethanesulfonic acid, hydroxyehtanesulfonic acid, benzenesulfonic acid, p-toluene sulfonic acid, cyclohexanesulfamic acid, salicyclic acid, p-aminosalicyclic acid, 2-phenoxybenzoic acid, 2-acetoxybenzoic acid, etc. In one preferred example, the compound of formula (I) is in the form of a fumarate salt (e.g., formoterol fumarate). In another example, the compound of formula (I) is in the form of a tartrate salt (e.g., formoterol tartrate or artformoterol tartrate).

The term “subject” or “patient” refers to an animal including the human species that is treatable with the methods of the present disclosure. The term “subject” or “patient” intended to refer to both the male and female gender unless one gender is specifically indicated. Accordingly, the term “subject” or “patient” comprises any mammal which may benefit from treatment of corneal injury or symptoms associated with corneal injury. Examples of a “subject” or “patient” include, but are not limited to, a human, rat, mouse, guinea pig, monkey, pig, goat, cow, horse, dog, cat, bird and fowl. In an exemplary embodiment, the patient is a human.

The phrase “pharmaceutically acceptable carrier” as used herein means a pharmaceutically acceptable material, composition or vehicle, such as a liquid or solid filler, diluent, carrier, solvent or encapsulating material, involved in carrying or transporting the subject agents from one organ (or one portion of the body) to another organ (or another portion of the body). Each carrier must be “acceptable” in the sense of being compatible with the other ingredients of the formulation. The pharmaceutical formulation contains at least one compound as set forth in the present method in combination with one or more pharmaceutically acceptable ingredients. For the clinical use of the methods of the present invention, the pharmaceutical composition of the invention is formulated into formulations suitable for the intended route of administration.

### II. Description of the Invention

The inventors of the present disclosure unexpectedly identify a series of compounds independently having the ability of suppressing corneal endothelial cells from undergoing apoptosis, thus these compounds are potential candidates for the development of medicaments for treating corneal injuries or corneal diseases.

### 1. The Compound of the Present Disclosure and Pharmaceutical Composition Comprising the Same

Accordingly, the first aspect of the present disclosure involves compounds or their pharmaceutically acceptable salts capable of suppressing corneal decompensation, accordingly, these compounds or their pharmaceutically acceptable salts may be suitable for treating corneal injury. Said compounds may be any one of a compound of Formula (I), Bhilawanol A, Encainide, Fenbutrazate, Levonordefrin, Permethrin, or 2,2,4,4-Tetrahydroxybenzophenone (THBP),

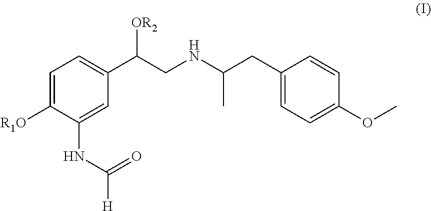

wherein in the compound of Formula (I),
R₁ is H, —SO₃H, or

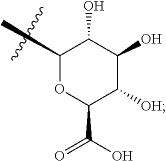

and
R₂ is H or

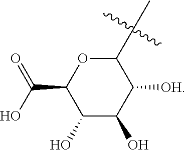 [SMILES: *C1O[C@H](C(=O)O)[C@@H](O)[C@H](O)[C@H]1O]

According to some embodiments, in the compound of Formula (I), R₁ and R₂ are independently H; accordingly, the compound has the structure of Formula (I)-1 (Formoterol),

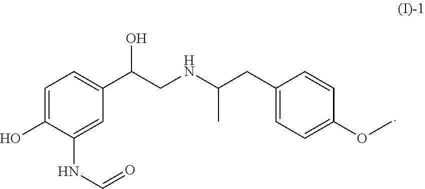 [SMILES: COc1ccc(CC(C)NCC(O)c2ccc(O)c(NC=O)c2)cc1]

In other embodiments, in the compound of Formula (I), R₁ is —SO₃H, and R₂ is H; accordingly, the compound has the structure of Formula (I)-2 (Formoterol Sulfate),

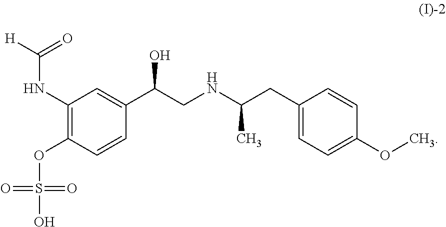 [SMILES: COc1ccc(C[C@@H](C)NC[C@H](O)c2ccc(OS(=O)(=O)O)c(NC=O)c2)cc1]

In further embodiments, in the compound of Formula (I), R₁ is H, and R₂ is

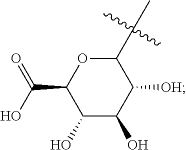 [SMILES: *C1O[C@H](C(=O)O)[C@@H](O)[C@H](O)[C@H]1O]

accordingly, the compound has the structure of Formula (I)-3 (Formoterol Benzyl Glucuronide, Formoterol BG),

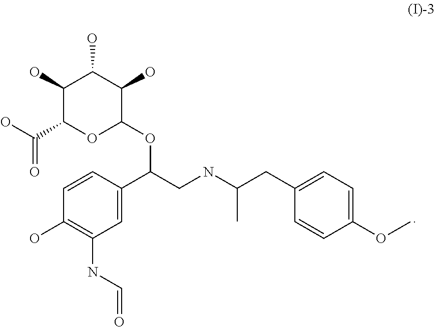 [SMILES: COc1ccc(CC(C)NCC(OC2O[C@H](C(=O)O)[C@@H](O)[C@H](O)[C@H]2O)c2ccc(O)c(NC=O)c2)cc1]

In still further embodiments, in the compound of Formula (I), R₁ is

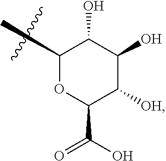

and R₂ is H; accordingly, the compound has the structure of Formula (I)-4 (Formoterol Phenolic Glucuronide, Formoterol PG),

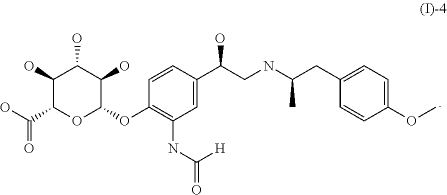 [SMILES: COc1ccc(C[C@@H](C)NC[C@H](O)c2ccc(O[C@@H]3O[C@H](C(=O)O)[C@@H](O)[C@H](O)[C@H]3O)c(NC=O)c2)cc1]

Preferably, the compound of formula (I) is in the form of a salt, such as fumarate or tartrate salts. In some embodiments, the compound of formula (I) is in the form of a fumarate salt (e.g., formoterol fumarate). In other embodiments, the compound of formula (I) is in the form of a tartrate salt (e.g., formoterol tartrate).

As would be appreciated, the compounds as listed above may be administered as a single compound to treat corneal injury, but are generally administered in the form of a pharmaceutical composition, which comprises one or more of the above compounds; and a pharmaceutically acceptable carrier. In some embodiments, the pharmaceutical composition may comprise two above-listed compounds (e.g., Formoterol and Bhilawanol A; Formoterol and Encainide; Bhilawanol A and Encainide; and the like). In some embodiments, the pharmaceutical composition comprises three above-listed compounds (e.g., Formoterol, Bhilawanol A, and Encainide; Formoterol, Levonordefrin, and THBP; Levonordefrin, THBP, and Bhilawanol A; Levonordefrin, THBP, and Encainide; and the like).

Optionally, the pharmaceutical composition comprising one or more compounds of the present disclosure can be administered alone or with other therapeutic drugs (e.g., an ophthalmological anti-infective agent). Suitable ophthalmological anti-infective agent that may be used with the pharmaceutical composition includes, but is not limited to, antibiotics (e.g., Chloramphenicol, Azidamfenicol, Tetracycline, Chlortetracycline, Oxytetracycline, Dihydrostreptomycin, Neomycin, Framycetin, Kanamycin, Amikacin, Tobramycin, Gentamicin, Netilmicin, Micronomicin, Erythromycin, Natamycin, Ampicillin, Benzylpenicillin, Polymyxin B, Tyrothricin, Rifamycin, or Fusidic acid); sulfonamides (e.g., Sulfamethizole, Sulfafurazole, Sulfadicramide, Sulfacetamide, or Sulfafenazol); antivirals (e.g., Idoxuridine, Trifluridine, Aciclovir, Interferon, Vidarabine, Famciclovir, Fomivirsen, or Ganciclovir); or other agents (e.g., Nitrofural, Bibrocathol, Resorcinol, Sodium borate, Hexamidine, Chlorhexidine, Sodium propionate, Ofloxacin, Norfloxacin, Ciprofloxacin, Dibrompropamidine, Propamidine, Picloxydine, Lomefloxacin, Povidone-iodine, Levofloxacin, or Gatifloxacin).

The pharmaceutical composition comprising one or more compounds of the present disclosure can be in any dosage form at the time of provision or storage, depending on their intended uses and the appropriate pharmaceutically acceptable carrier selected to achieve such uses. The pharmaceutical composition comprising one or more compounds of the present disclosure may have a dosage form of solid, semi-solid or liquid, gel, cream, or the like. The pharmaceutical composition comprising one or more compounds of the present disclosure can be generally administered as an oral administration agent such as tablet, capsule, powder, granule, pill, syrup or the like, eye drop, ophthalmic ointment, percutaneous absorbent, ophthalmic injection, or spray. Among these, external preparations for the eyeball including eye drop, ophthalmic ointment, ophthalmic injection, and spray, are preferable, and eye drop and spray are more preferable because they can be easily administered locally to the eye with minimal invasiveness.

As the ophthalmic ointment, a conventional base such as white petrolatum, liquid paraffin, and the like, can be prepared. Eye drops can be prepared by using isotonic agents (such as sodium chloride, potassium chloride, glycerol, propylene glycol, and the like); buffering agents (such as sodium phosphate, sodium acetate, sodium borate, sodium carbonate, and the like); surfactants (such as polyoxyethylene sorbitan fatty acid ester, polyoxyl stearate 40, polyoxyethylene polyoxypropylene glycol, polyoxyethylene castor oil, polyoxyethylene hydrogenated castor oil, and the like); stabilizers (such as disodium edetate, sodium citrate, and the like); antiseptics (such as benzalkonium chloride, sorbic acid, methyl parahydroxybenzoate, and the like); thickening agents (such as methylcellulose, hydroxymethylcellulose, polyvinylpyrrolidone, and the like); antioxidants (such as ascorbic acid, tocopherol, and the like); and the like as necessary. The pH may be in the range acceptable for ophthalmological preparations, and is preferably in the range of 4 to 8. When adjusting the pH, hydrochloric acid, phosphoric acid, citric acid, sodium hydroxide, potassium hydroxide, sodium carbonate, and the like, can be used.

In general, the one or more present compounds are present in the pharmaceutical composition at a level of about 0.01% to 99% by weight, based on the total weight of the pharmaceutical composition. In some embodiments, the one or more present compounds are present at a level of at least 0.1% by weight, based on the total weight of the pharmaceutical composition. In certain embodiments, the one or more present compounds are present at a level of at least 1% by weight, based on the total weight of the pharmaceutical composition. In other embodiments, the one or more present compounds are present at a level of at least 5% by weight, based on the total weight of the pharmaceutical composition. In still other embodiments, the one or more present compounds are present at a level of at least 10% by weight, based on the total weight of the pharmaceutical composition. In still yet other embodiments, the one or more present compounds are present at a level of at least 25% by weight, based on the total weight of the pharmaceutical composition.

### 2. The Treatment Method

Accordingly, the present disclosure also encompasses a method for treating a corneal injury in a subject in need thereof. The method comprises administering an effective amount of the present pharmaceutical composition to the subject.

The corneal injury treatable by the present compound, the pharmaceutical composition, and/or the method is caused by intraocular surgery, but the present invention is not limited thereto. As the present compound suppresses cell apoptosis and facilitates wound healing, thus the present pharmaceutical compound is useful for treating any corneal injuries involving the process described above, regardless of their locations or the causes therefrom.

According to the present method, the present pharmaceutical composition is administered to the subject in the amount of about 0.1 pg/kg to 100 mg/kg body weight of the subject, such as 0.1, 0.2, 0.3 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1,000 pg/kg (=1 ng/kg), 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1,000 ng/kg (=1 μg/kg), 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1,000 ag/kg (=1 mg/kg), 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg body weight of the subject; preferably, about 10 μg/kg to 1 mg/kg body weight of the subject; more preferably, about 0.1 ng/kg to 1 ag/kg body weight of the subject; even more preferably, about 2 ng/kg to 30 ng/kg body weight of the subject. In some working examples, the present pharmaceutical composition is administered to the subject in the amount of about 2.748 ng/kg (for Levonordefrin), 3.75375 ng/kg (for THBP), 5.166 ng/kg (for Formoterol), 6.3675 ng/kg (for Formoterol Sulfate), 7.8075 ng/kg (for Formoterol PG), 7.8075 ng/kg (for Formoterol BG), 23.8875 ng/kg (for Bhilawanol A), 5.5125 ng/kg (for Fenbutrazate), 5.8695 ng/kg (for Permethrin), and 5.2875 ng/kg (for Encainide). The dose can be administered in a single aliquot, or alternatively in more than one aliquot. The skilled artisan or clinical practitioner may adjust the dosage or regime in accordance with the physical condition of the patient or the severity of the diseases.

The skilled artisan or health practitioner may adjust the dosing regimen of the present pharmaceutical composition in accordance with various factors, such as age, gender, weight, and other treatments (if any). For example, the present pharmaceutical composition may be administered to the subject 1-7 times per week (e.g., 1, 2, 3, 4, 5, 6 or 7 times per week) for 1, 2, 3, 4 or more consecutive weeks. Alternatively, the present pharmaceutical composition may be administered to the subject 1-10 times (e.g., 1, 2, 3, 4, 5, 6, 7, 8, 9 or 10 times) for every 2 weeks, every 4 weeks, every 5 weeks, every 6 weeks, every 7 weeks, every 8 weeks, every 9 weeks, or every 10 weeks; or once every month, every 2 months, or every 3 months, or longer. Preferably, the present pharmaceutical composition is administered to the subject daily for at least 1 day, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days. More preferably, the present pharmaceutical composition is administered to the subject daily for at least 28 days (i.e., 4 weeks). Even more preferably, the present pharmaceutical composition is administered to the subject daily for at least 14 days (i.e., 2 weeks). According to one working example of the present disclosure, the present pharmaceutical composition is administered only once to the subject daily for the entire course of treatment (i.e., 2 days) so as to produce the therapeutic effect.

Optionally, the present method further comprises administering an ophthalmological anti-infective agent to the subject, in combination with administering the present pharmaceutical composition. Exemplary ophthalmological anti-infective agents are as described above, which may be formulated into a pharmaceutical composition or a pharmaceutical kit along with the compound used in the present method. The ophthalmological anti-infective agent may be administered to the subject prior to, in conjunction with, or subsequent to application of the present pharmaceutical composition to the subject at different doses, time intervals, via the same or different routes. The doses and time intervals may vary with factors such as described above, and are dependent on the professional considerations of the practitioner; and the routes may be via oral, enteral, buccal, nasal, transdermal, transmucosal, intravenous, intraperitoneal, intraarterial, intracutaneous, subcutaneous, intramuscular, and intracameral routes. Preferably, the administration route suitable for use in the present method is the intracameral route, in which the active compound of the present disclosure is administered intracamerally in the form of an eye drop or by direct intracameral injection.

Basically, the subject treatable by the present method is a mammal; preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Materials and Methods

### 1. Drug Preparation

Levonordefrin (Sigma), Formoterol (Santa-Cruz), Formoterol Sulfate (SynZeal), Formoterol BG (SynZeal), Formoterol PG (SynZeal), 2,2,4,4-Tetrahydroxybenzophenone (THBP) (Sigma), Permethrin (Sigma), Bhilawanol A (Sigma), Fenbutrazate (Santa-Cruz), or Encainide (Sigma) was dissolved in DMSO (Sigma) to reach a final concentration of 10 mM as a stock solution, which was then stored at −20° C. for later use. For preparing working solutions, each stock solution was diluted with the balanced salt solution (BSS) (Alcon) to reach a desired working concentration prior to intracameral injection.

### 2. Mouse Strain

nT/nG transgenic mice (Jax #023035; the Jackson Laboratory), having a Cre-reporter allele comprising a cell nuclear-targeted tandem dimerTomato (tdTomato; an orange fluorescent protein) and an enhanced green fluorescent protein (EGFP) (Prigge, Wiley et al., 2013; “Nuclear double-fluorescent reporter for in vivo and ex vivo analyses of biological transitions in mouse nuclei.”), were used in the present study for investigating the cell density and the healing process in corneal endothelium. The mice were characterized in that, prior to Cre recombination, widespread fluorescence expression of tdTomato (localized to the cell nucleus) was observed; whereas following Cre recombination, nuclear-localized green fluorescence was detected. All procedures on mice were approved by and performed in accordance with the Institutional Animal Care and Use Committees of National Taiwan University.

### 3. Animal Live Imaging

For live image acquisition, mice were anesthetized by intramuscular injection of 50-80 mg/kg Zoletil (Virbac). The corneal surface was anesthetized with 0.4% oxybuprocaine hydrochloride (Sigma) in saline. An eye holder was used to minimize artifacts during intravital imaging, as previously described (Wu, Wang et al., 2019; “Intravital multiphoton microscopic imaging platform for ocular surface imaging.”). Intravital imaging was performed using a laser wavelength of 880 nm for tdTomato excitation and a laser power of approximately 35 mW at the sample plane to optimize the image quality without photobleaching. A water-immersion 20×/NA1.0 objective lens (Olympus) was used to focus the laser light onto the eyes, after the laser light was reflected by the main dichroic mirror (Semrock). The tdTomato signals were spectrally separated using a bandpass filter of 585/40 nm (Semrock). To prevent dryness of the ocular surface during imaging, an eye gel (Vidisic Gel, Dr. Gerhard Mann Chem-Pharm) with a refractive index of 1.338 was used as an immersion medium on the cornea. Image stacks along the z-axis with a step size of 1 μm were acquired, and the dimensions of the images acquired in this study were 512×512 pixels.

### 4. Corneal Endothelial Cell Ablation

To eliminate the corneal endothelial cells, the multiphoton femtosecond laser was employed to induce cell ablation due to its ability to precisely control cell ablation in time and space with minimal injury made to adjacent tissues (Rompolas, Deschene et al., 2012; “Live imaging of stem cell and progeny behaviour in physiological hair-follicle regeneration.”). Once the corneal endothelial cells needed to be removed were identified, laser ablation was performed on the selected area (50 μm×50 μm) with a 880 nm laser wavelength and a 500 mW laser power in a single scan, with repeatedly adjusting the laser along the z-axis to achieve optimal results.

### 5. Immunofluorescence Assay

After laser ablation, the enucleated eyeballs were fixed in 4% paraformaldehyde solution (PFA) solution (Santa-Cruz) at 4° C. overnight, followed by washing with PBS (Protech). The corneas were then dissected from the eyeballs under dissecting microscope and blocked with 5% BSA (Bio Basic)/PBS containing 1% Triton-X 100 (VWR) at 4° C. overnight. The corneas were incubated with N-cadherin antibodies (1:200, Cell Signaling) in blocking buffer at 4° C. overnight and stained with conjugated secondary antibodies AlexaFluor 647 (1:500, Jackson ImmunoResearch) at 4° C. overnight. Lastly, the apoptotic cells were detected by the TUNEL kit (Promega) and the stained corneas were mounted by VECTASHIELD Antifade Mounting Medium (Vector). All confocal images were acquired by SP8 confocal microscope (Leica).

### 6. Intracameral Drug Delivery

Following laser ablation, the aforementioned drugs were intracamerally injected to the cornea of the mice immediately using a customized needle attached to a 5 μl syringe (Hamilton). To avoid the motion, a cotton swab was used to prop the eyeball during the intracameral injection above on the peripheral region of the cornea. Each eye was slowly applied with a single dose of 0.5 μl different drugs. A non-toxic fluorescein dye was used concurrently to confirm drugs constantly being preserved in aqueous humor without leak.

### 7. Cell Density Quantification

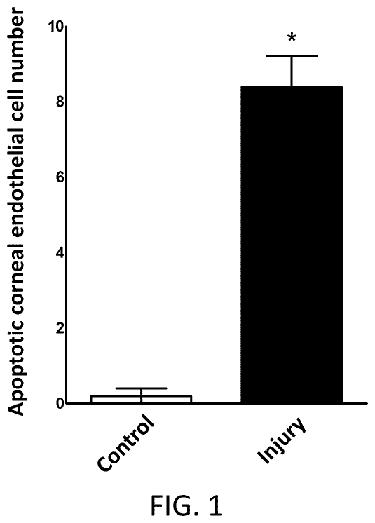

The mice were anesthesia and sacrificed on day 2 after laser ablation with the indicated treatments administered (as shown in FIGS. 1-3), and their eyes were enucleated and fixed in 4% paraformaldehyde (PFA) at 4° C. overnight. After dissecting the cornea from the eyeball, the cornea was cut into a four-petal shape, placed on glass slides, mounted with a mounting medium (Vector), and immobilized by using a coverslip. All the fluorescent images were acquired by a SP8 confocal microscope (Leica), with steps set at 1 μm per z-section for a 20× objective. The cell nuclear ware manually segmented by using spot plugin in Imaris (Oxford instruments). For each sample, a circle area with a diameter of 300 μm was selected to count the cell numbers, so as to quantify the cell density in different drug treatments.

### 8. Statistical Analyses

All the experimental data were performed by Prism and represented as means±SEM. The statistical significance of difference between the experimental groups was evaluated by Student's t test. P values<0.05 were considered statistically significant.

### Example 1 the Present Compounds Improved Healing of the Laser-Injured Cornea

### 1.1 Establishing a Laser-Injured Cornea Animal Model

In the present study, the effects of the present compounds on the injured cornea were investigated. To this end, an animal model of corneal injury using a laser to simulate injuries caused by external forces, such as ophthalmic surgeries, was first established herein in accordance with the procedures as described in the section of “Materials and Methods.” The laser ablation treatment caused the loss of the corneal endothelial cells in the treated area, as evidenced by the absence of N-cadherin staining (data not shown). Further, the TUNEL staining of the injured cells exhibited a significant increase, suggesting that the corneal endothelial cells underwent apoptosis after treated with laser (FIG. 1).

### 1.2 Intracameral Injection of Drugs

The test compound(s) was/were intracamerally administered to the mice following laser ablation of the cornea, and then the corneal opacity was initially evaluated via gross images of the cornea. It was observed that the corneas were transparent before and after laser ablation, while there were approximately-sized holes with a diameter of 0.1-0.2 mm appeared immediately after intracameral injection (data not shown). On Day 2, only the BSS group exhibited mild inflammation and irritation in the cornea. By contrast, all the tested drugs, including Levonordefrin, Formoterol, Formoterol Sulfate, Formoterol BG, Formoterol PG, THBP, Permethrin, Bhilawanol A, Fenbutrazate, and Encainide, maintained the ocular surface of the eye moist and transparent, while the Fenbutrazate group showed slightly hazy spot formation. All the aforementioned observations were summarized in Table 1. Collectively, all the tested drugs were found to be suitable for intracameral injection, without causing appreciable adverse effects on the ocular surface.

TABLE 1
Details of the laser-injured cornea after
treatment with the test compounds

| | Observation | | |
|---|---|---|---|
| Treatment | Opacity | Edema | Ulcer formation |
| Control | No | No | No |
| BSS | Slight opacity | Mild edema | Slight ulcer |
| | | | formation |
| Levonordefrin | No | No | No |
| Formoterol | No | No | No |
| Formoterol Sulfate | No | No | No |
| Formoterol BG | No | No | No |
| Formoterol PG | No | No | No |
| THBP | No | No | No |
| Permethrin | No | No | No |
| Bhilawanol A | No | No | No |
| Fenbutrazate | Slight hazy | No | No |
| Encainide | No | No | No |

### 1.3 Promoting Corneal Wound Healing

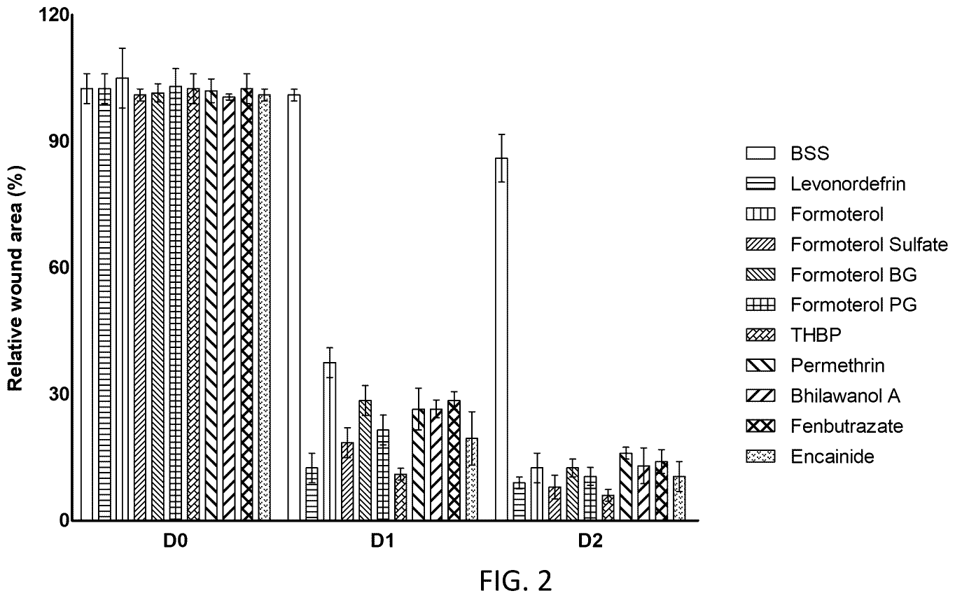

The efficacy of the tested drugs in promoting the corneal wound healing was investigated by a multiphoton microscopy analysis, in which changes in the wound site following drug injection was tracked. On Day 1 after laser ablation, it was found that the corneal endothelial cells in the BSS group could not seal the wound defects, and the compromised integrity of the corneal endothelium continued until Day 2 (FIG. 2). By contrast, intracameral injection of 6 μM (27.48 ng/kg) Levonordefrin and 6 μM (37.5375 ng/kg) THBP accelerated wound healing and sealed the defects on Day 1; whereas intracameral injection of 6 μM (51.66 ng/kg) Formoterol, 6 μM (63.675 ng/kg) Formoterol Sulfate, 6 μM (78.075 ng/kg) Formoterol PG, 6 μM (78.075 ng/kg) Formoterol BG, 30 μM (238.875 ng/kg) Bhilawanol A, 6 μM (55.125 ng/kg) Fenbutrazate, 6 μM (58.695 ng/kg) Permethrin, and 6 μM (52.875 ng/kg) Encainide promoted wound healing on Day 2. The aforementioned drugs promoted wound healing albeit to varying extents; they all facilitated the corneal endothelial wound healing within two days.

1.4 Protecting Corneal Endothelial Cells from Injuries

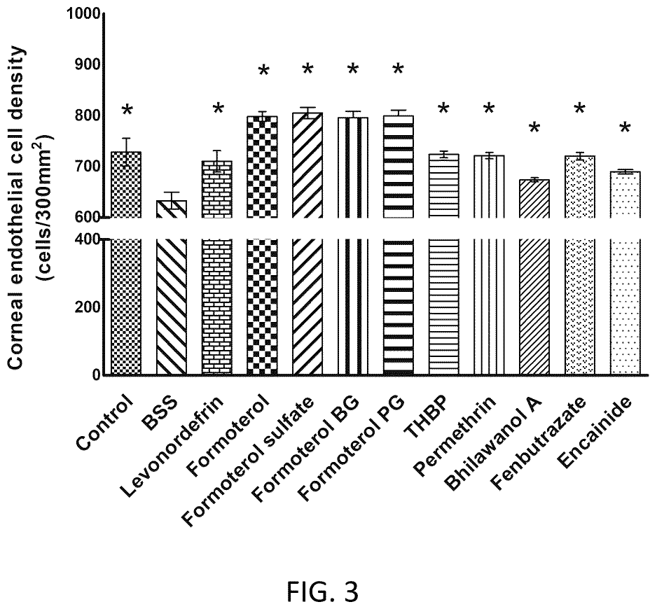

The protective effect of the present compound(s) on the corneal endothelial cells against injuries was evaluated by counting the cell number and/or the density of the corneal endothelial cells, instead of the keratocytes in corneal stroma or the other cell populations in trabecular meshwork. The results were shown in FIG. 3, and Table 2. It was found that the drug protected the corneal endothelial cells in the order of, Formoterol Sulfate>Formoterol PG>Formoterol BG>Formoterol>THBP>Permethrin>Fenbutrazate>Levonordefrin>Encainide>Bhilawanol A. Taken together, the data demonstrated that the drugs as provided herein have the potential to promote primary wound healing and protect the corneal endothelial cells from injuries.

TABLE 2
The corneal endothelial cell density values
for the different treatment groups

| | Cell Density (cell number/ |
|---|---|
| | the area of a circle with a diameter of |
| Treatment | 300 μm) |
| Control | 728.5 ± 55.01 |
| BSS | 633 ± 33.7 |
| Levonordefrin | 710.5 ± 41.7 |
| Formoterol | 743.3 ± 14 |
| Formoterol Sulfate | 805.25 ± 22.3 |
| Formoterol BG | 796 ± 24.9 |
| Formoterol PG | 799.5 ± 23.56 |
| THBP | 724 ± 12.8 |
| Permethrin | 721.8 ± 12.9 |
| Bhilawanol A | 674.3 ± 8 |
| Fenbutrazate | 720.8 ± 14.9 |
| Encainide | 690 ± 8.8 |

In summary, the present disclosure provides a novel method for corneal injuries by use of one or more drugs described above to enhance corneal wound healing and prevent corneal decompensation, which in turns reduces the need for corneal transplantation surgery.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1-9. (canceled)

10. A method for treating corneal injury in a subject comprising administering to the subject an effective amount of a compound of Formula (I) or a pharmaceutically acceptable salt thereof, 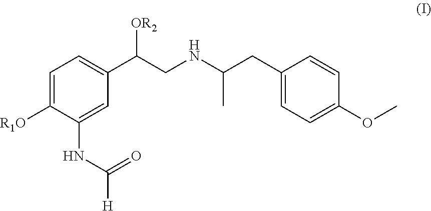
wherein
R₁ is H, —SO₃H, or 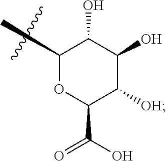
and
R₂ is H or

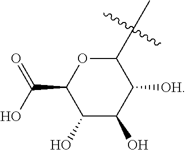 [SMILES: *C1O[C@H](C(=O)O)[C@@H](O)[C@H](O)[C@H]1O]

11. The method of claim 10, wherein in the compound of Formula (I),
R₁ and R₂ are independently H;
R₁ is —SO₃H, and R₂ is H;
R₁ is H, and R₂ is 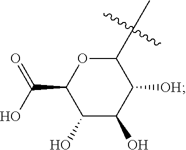 [SMILES: *C1O[C@H](C(=O)O)[C@@H](O)[C@H](O)[C@H]1O]
or
R₁ is 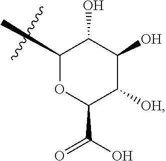
and R₂ is H.

12. The method of claim 11, wherein the compound of Formula (I) is in the form of a tartrate salt or a fumarate salt.

13. The method of claim 10, wherein the corneal injury is a corneal endothelial injury.

14. The method of claim 10, wherein the corneal injury is caused by intraocular surgery.

15. The method of claim 10, wherein the corneal injury is resulted from apoptosis during wound healing.

16. The method of claim 10, wherein the compound is administered to the subject in the amount of about 0.1 ng/kg to 1 μg/kg.

17. The method of claim 10, wherein the compound is administered to the subject intracamerally.

18. The method of claim 17, wherein the compound is administered in the form of an eyedrop or by direct injection.

19. The method of claim 10, wherein the subject is a human.

20. The method of claim 10, further comprising administering an ophthalmological anti-infective agent to the subject prior to, in conjugation with, or after administering of the compound.

21. The method of claim 20, wherein the ophthalmological anti-infective agent is selected from the group consisting of Chloramphenicol, Azidamfenicol, Tetracycline, Chlortetracycline, Oxytetracycline, Dihydrostreptomycin, Neomycin, Framycetin, Kanamycin, Amikacin, Tobramycin, Gentamicin, Netilmicin, Micronomicin, Erythromycin, Natamycin, Ampicillin, Benzylpenicillin, Polymyxin B, Tyrothricin, Rifamycin, Fusidic acid, Sulfamethizole, Sulfafurazole, Sulfadicramide, Sulfacetamide, Sulfafenazol, Idoxuridine, Trifluridine, Aciclovir, Interferon, Vidarabine, Famciclovir, Fomivirsen, Ganciclovir, Nitrofural, Bibrocathol, Resorcinol, Sodium borate, Hexamidine, Chlorhexidine, Sodium propionate, Ofloxacin, Norfloxacin, Ciprofloxacin, Dibrompropamidine, Propamidine, Picloxydine, Lomefloxacin, Povidone-iodine, Levofloxacin, and Gatifloxacin.
